(19) United States
(12) Patent Application Publication
(10) Pub. No.: US 2007/0167998 A1
(43) Pub. Date: Jul. 19, 2007

(54) Phototherapy device and method

(76) Inventor: Yves Loones, Precigne (FR)

Correspondence Address: LOWE HAUPTMAN GILMAN & BERNER, LLP, 1700 DIAGNOSTIC ROAD, SUITE 300, ALEXANDRIA, VA, 22314, US

(21) Appl. No.: 10/589,280

(22) PCT Filed: Feb. 10, 2005
(86) PCT No.: PCT/EP05/50596

§ 371 Date: Feb. 12, 2007

(30) Foreign Application Priority Data
Feb. 11, 2004 (FR) 04 01325

(51) Int. Cl. A61N 5/06

(52) U.S. Cl. 607/88; 607/90

(57) ABSTRACT

Phototherapy equipment comprising a light source (5, 49), a light guide (6, 46) suitable for directing the light to the inlet of a terminal pen (4, 50) for projecting a light beam onto living tissues and comprising a plate (28, 70) at the outlet of the polarizer (25, 65) arranged to give the light a defined, clockwise or counterclockwise direction (D, L) of polarization.

The invention relates to phototherapy equipment used notably in therapeutic methods for treatment of skin disease or other diseases at greater depth.

It also relates to an equipment and a method of phototherapy used in the food-processing industry.

It is known that phototherapy with light of high luminosity is applicable in a certain number of disorders such as winter depression, selective sleep disorders, interruption of circadian rhythm, etc.

However, phototherapy can also be employed effectively in more severe pathologies, notably in the area of dermatology. Intensive exposure of patients suffered from acne to a combination of two monochromatic lights proved more effective and three times faster than any other existing treatment. Porphyrin, which is produced, normally, by one of the commonest bacteria responsible for acne, Propionibacterium acnes, is converted to a poison for the bacterium, which destroys it. By using dynamic phototherapy, so-called because it consists of using a photosensitizing substance that is a precursor of the porphyrins, and inducing a phototoxic reaction by irradiating the treated zone with light of a suitable wavelength, significant results are obtained in the great majority of superficial carcinomas.

Moreover, polarized light permits better healing owing to its biostimulating effect, like laser light, which is also known to have analgesic, anti-inflammatory and antiedemic effects.

Photons of light, by electrochemical stimulation of the cellular ions, and the electromagnetic radiation of light, by an effect of biological resonance and the intrinsic variation of their electromagnetic field, can have a beneficial effect on living tissues which store, notably by their DNA (deoxyribonucleic acid) and their RNA (ribonucleic acid), electromagnetic energy released on fields of oscillations, interacting at depth on the cellular molecules by propagation of their intrinsic radiation.

It is therefore clearly advantageous to find and investigate new treatments of this type. There are at present few instruments available for phototherapy that are capable of irradiating biological tissues with photons of light, whether it is coherent or not, and whether it is polarized or not. They do not allow to explore all the possibilities that can be envisaged.

The present invention is based notably on the following principle: the importance of the direction of polarization of light acting on asymmetric (e.g. chiral) molecules is comparable to the importance of the orientation of the inductive magnetic field of a primary winding on an asymmetric armature (e.g. magnetically polarized).

With selection of the color of the light in relation to the treatment to be applied, the invention additionally permits the orientation of polarization of this light to be selected, so as to adapt this orientation—dextrorotatory or levorotatory—to the right or left chirality of the molecules to be treated, depending on the treatment.

The invention relates to phototherapy equipment having at least one light source, a light guide adapted for carrying the light to the inlet of a terminal pen for projecting a light beam onto living tissues, characterized in that it comprises at least one plate at polarizer outlet arranged to give the light a particular direction (D, L) of polarization, clockwise or counterclockwise.

The plate can be a quarter-wave plate or a half-wave plate.

The plate is for example positioned according to two positions of use.

It can be positioned at approximately 45° to the left or at approximately 45° to the right of a neutral position.

The two positions are obtained, for example, under the action of a micro-motor operating with clockwise (D) or counterclockwise (L) rotation.

The light guide is, for example, an optical fiber cable.

The pen includes for example an iris.

The equipment includes, for example, a barrel provided with filters of different wavelengths connected to a drive unit, the barrel being arranged at the outlet of the light source.

The polarizer can be a circular polarizer.

The polarizer can also be an elliptical polarizer.

The light source is, for example, a halogen or xenon lamp equipped with monochromatic filters.

The light source can be a laser diode.

The light source is, for example, made up of a set of laser diodes of different colors.

The invention also relates to a method of cosmetic treatment of tissues of biological cells by phototherapy comprising the irradiation of tissue with incoherent and/or coherent polarized monochromatic light, characterized in that the wavelength to be used is selected and the direction of polarization of the light is determined in order to adapt this dextrorotatory or levorotatory orientation to the right or left chirality of the molecules depending on the treatment to be applied.

According to another variant of application, the invention relates to a method for food-processing industry use for treating tissues of biological cells by phototherapy, comprising the irradiation of tissue with incoherent and/or coherent polarized monochromatic light, characterized in that the wavelength to be used is selected and the direction of polarization of the light is determined in order to adapt this dextrorotatory or levorotatory orientation to the right or left chirality of the molecules depending on the treatment to be applied.

A quarter-wave plate or a half-wave plate is used.

The equipment according to the invention can also be applied to the treatment of biological cells by phototherapy, comprising the irradiation of tissue with incoherent and/or coherent polarized monochromatic light, characterized in that the wavelength to be used is selected and the direction of polarization of the light is determined in order to adapt this dextrorotatory or levorotatory orientation to the right or left chirality of the molecules, depending on the treatment to be applied.

The invention notably makes it possible to apply phototherapy that is more precise than in the prior art, by irradiation with light that stimulates or inhibits the treated tissues, because of being closer to the bio-electronic “operating point” of the molecules in their cells, and which can be called “gyro-chromato-biotherapy”.

Other characteristics and advantages of the present invention will become clearer on reading the description of an example of application, which is given for purposes of illustration and is in no way limiting, together with the diagrams, showing:

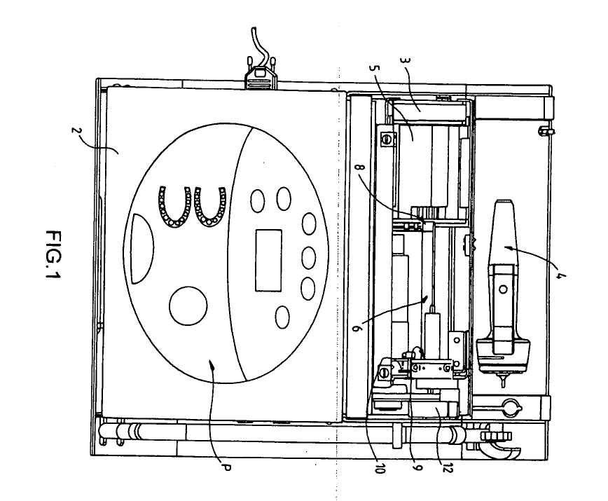

FIG. 1, a perspective view of an example of phototherapy equipment according to the invention,

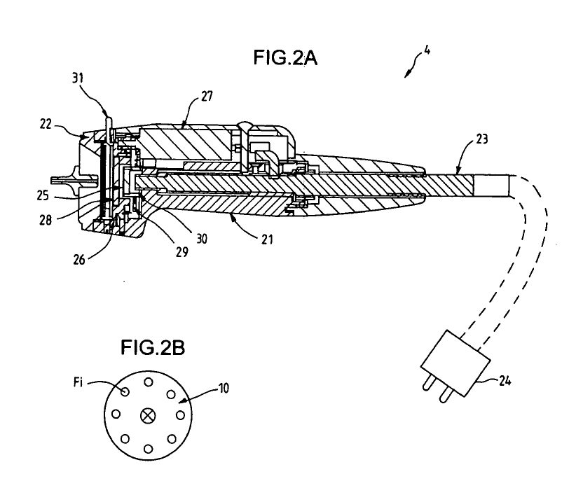

FIG. 2A: a cross-sectional view of the pen of the phototherapy equipment of FIG. 1, and FIG. 2B: a view of the barrel carrying the filters,

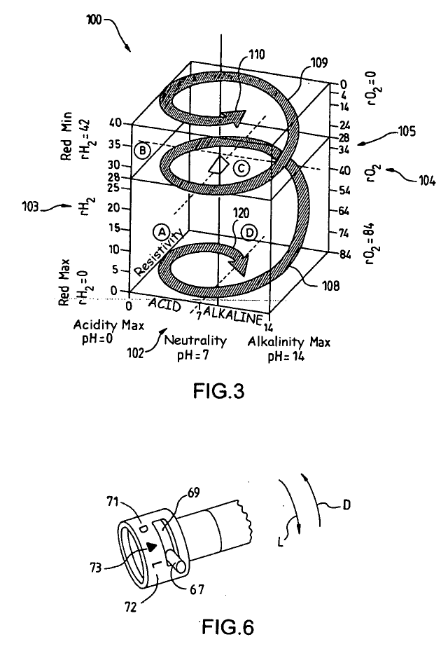

FIG. 3, a three-dimensional bioelectronic diagram of application of the method of phytotherapy,

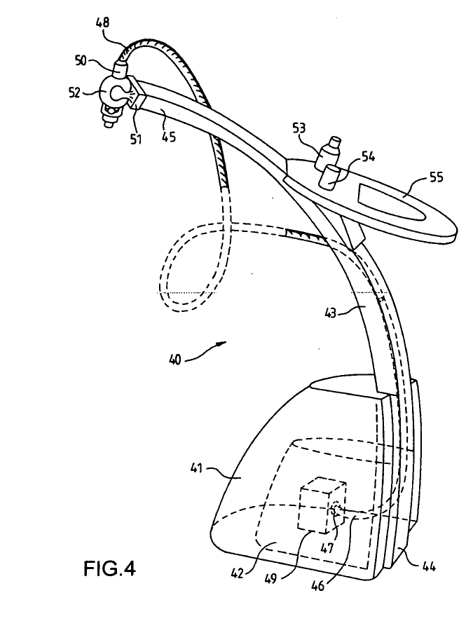

FIG. 4, a perspective view of another variant of equipment according to the invention,

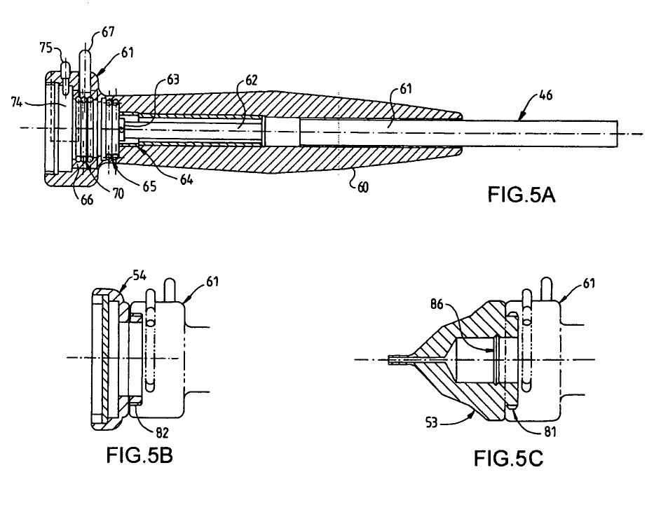

FIGS. 5A, 5B, 5C: cross-sectional diagrams of the pen of the phototherapy equipment,

FIG. 6, the sliding control button on the pen for actuating the quarter-wave plate.

The invention notably makes use of various characteristics displayed by biological molecules and structures undergoing treatment, for example:
certain bioelectronic potentials, namely a magnetic factor, the pH, a redox factor, rH₂ (or rO₂), and a factor of electrical resistivity p,
nonlinear magneto-optical properties and effects (Kerr effect) due to the anisotropy of some of them,
functions of the “helical waveguide” type that they may possess, notably twisted nematic biological structures such as cholesterol liquid crystals or certain phospholipids.

These last-mentioned waveguide functions make possible, in accordance with the nonlinear magneto-optical properties, the preferential propagation (without notable loss of charge) of dextrorotatory or levorotatory polarized electromagnetic radiation. Thus, the direction of induction will be mainly electrical (rH₂ or rO₂) or magnetic (pH) depending on the choice of wavelengths and their direction of polarization. For example, for a dextrorotatory magnetic stimulation there may be a corresponding levorotatory electrical response of the biological medium.

These characteristics appear to be relatively well established notably for molecules of DNA or of RNA.

Referring to FIG. 1, the phototherapy equipment 1 comprises notably a control and operating unit 2, a lamp unit 3, a pen 4 shown in detail in FIG. 2A and connected to the lamp unit by means of an optical fiber, for example.

The control and operating unit 2 comprises for example a processor P, designed for selecting a wavelength according to the treatment selected and for carrying out the various stages employed by the method.

Lamp unit 3 notably comprises a source light 5, for example a xenon lamp, a glass bar 6 notably having the function of conducting the light to the outlet 7 of the lamp unit.

Infrared filters are arranged at either end of the glass bar, 8, 9. The light used is for example incoherent and/or coherent monochromatic light.

A barrel 10 (FIG. 2B) provided with several filters Fi of different wavelengths is arranged in front of the outlet, shown but not referenced in the diagram, of the glass bar. The barrel is operated by a barrel drive unit 11 connected to the control and operating unit 2.

The light emitted from the lamp unit passes through the barrel and is then conducted to pen 4, connected to the lamp unit at the outlet 12 of the optical fiber 23.

Referring to FIG. 2A, the pen 4 has a sleeve 21 and, in the extension of this sleeve, a head 22. The sleeve and the head form a rectilinear light pipe, described in more detail below.

An optical fiber 23 with connector 24 is arranged inside sleeve 21. The fiber notably has the function of guiding the light of wavelength selected by the barrel, to the inlet of a polarizer 25 fixed to the inside wall of the sleeve by means of sealing rings, not identified in the drawing for simplicity. Barrel 10 is notably provided with filters Fi (FIG. 2B) of different wavelengths, selected in relation to the various pathologies to be treated. The barrel can be rotated by the drive unit in order to select the appropriate filter for the treatment.

At the outlet of polarizer 25, a collar 26 connected to a micro-drive unit 27 is arranged in the head. The collar can be rotated in the two directions of rotation L (levorotatory) and D (dextrorotatory).

A quarter-wave or half-wave plate 28 is fixed in the collar 26 for example by two sealing rings, not referenced in the drawing. Plate 28 filters the light leaving the polarizer. An electronic detection card 29 provided notably with sensors of the position of the plate is integral with the pen by means of a card holder 30. This card is connected to the motor and to the processor of the control and operating unit.

At the position marked D (FIG. 6, for example) on the head of the pen, the plate 28 gives right-polarized light, and at the position marked L on the head of the pen, left-polarized light is obtained. At the middle position, the polarized light at the outlet of the polarizer 25 is unchanged.

The pen also includes an iris 31 arranged at the outlet of the quarter-wave plate 28. The iris can be adjusted, for example by a sliding button 32 owing to an opening provided in the head, according to the two directions of rotation L and D. Rotation of the iris makes it possible to focus the emitted light beam according to the treatment requirements.

The drive unit and plate-holding collar are connected by mechanical means such as a gear-wheel system shown in the diagram.

The reducing motor is controlled by processor P.

To treat a living tissue by phototherapy, we first of all consider the biological cells that are to be treated and then apply irradiation with incoherent and/or coherent, monochromatic polarized light.

The living tissues can be tissues of vegetable organisms. It is also possible to apply the invention to the treatment of salmonella in eggs.

FIG. 3 shows a “bioelectronic” diagram 100 of the electromagnetic state of the cells to be treated, with the pH of the biological medium on the abscissa 102, and the electrical resistivity ρ of this medium on the ordinate, and on the vertical axis, rH₂ in the ascending direction 103, and rO₂ in the opposite direction 104. These last two parameters rH₂ and rO₂ are connected by the relation:
rO₂=2.rH₂−84

These data characterizing the biological medium are coefficients of ionic, protonic or electronic concentration, and are therefore dimensionless.

The pH scale extends from 0, indicating a medium of maximum acidity, to 14, which indicates a medium of maximum alkalinity. A pH of 7 indicates a neutral medium.

The scale of rH₂ ranges from 0, indicating a medium with low concentration of negative particles, and therefore very reducing, to 40, indicating a medium that is barely reducing, with neutrality at 28. The reverse is true for the oxidizing power, for the inverted scale of rO₂. Thus, a medium is reducing if its rH₂ is less than 28, and is oxidized, therefore oxidizing, if it is greater than 28.

Four cases can be distinguished, according to the bioelectronic theory of Prof. Vincent (Traité de Biologie électronique Dr—Ing J. A. Giralt-Gonzalez. Publ. Roger Jollois 1993)
A: the medium is acidic and reducing, favorable to development,
B: the medium is acidic and oxidized, favorable to preservation,
C: the medium is alkaline and oxidized, favorable to degradation,
D: the medium is alkaline and reducing, favorable to putrefaction.

In each of these cases, the biological medium reacts differently to the electromagnetic waves from the phototherapy equipment by returning bioelectronic energy to its environment.

The energy returned to the environment is assumed to depend not only on the color of the incident light, but also on the direction, dextrorotatory or levorotatory, of its polarization.

Following the same principle, DNA is assumed notably to return energy by dextrorotatory or levorotatory electromagnetic radiation, according to the cases expounded above.

As an example, consider a bioelectronic configuration as shown in FIG. 3 by a spiral 108, and more precisely, in cases B and C, by its portion defined between points 109 and 110, in which the energy returned is more easily levorotatory; we will choose to project left-polarized red or orange light to stimulate the return, or right-polarized light of another color, if we wish to inhibit it.

Conversely, in cases A and D, staying consistent with the above cases, at point 120 of the spiral we will choose right-polarized violet or blue light to stimulate this return.

The equipment is suitable, for example, for use:
on the skin itself, for local action on the tissues, on the surface or at depth,
on acupuncture points, and/or on metameric and reflexology zones for a more general action on the organism,
in ocular action.

To summarize, the method comprises notably the following stages:
a) select the wavelength to be used notably in relation to the pathology to be treated,
b) determine the direction of polarization of the light in relation to the bioelectronic affinity of the living cells to be treated, using for example the diagram in FIG. 3,
c) position, by means of the drive unit, the quarter-wave plate or half-wave plate in the desired position,
d) irradiate the tissue to be treated.

The time of exposure of the tissue to the light and/or the sequencing of the stages is notably under the control of processor P.

The method can be applied notably in the agricultural and food sector and for cosmetic treatments.

FIG. 4 shows another embodiment of the phototherapy equipment.

The phototherapy equipment 40 comprises a base 41, containing a light box 42 and supporting an arm 43 having two ends 44 and 45.

A fiber-optic cable 46 passes, for example, at least partially through arm 43. Cable 46, which has two ends 47 and 48, is connected on the one hand to a light source 49 arranged in the light box and on the other hand to a terminal pen 50 at its end 48.

A head 51 with a swivel joint 52 is integral with end 45 of arm 43, which also supports a tray 55 for storing tips 53, 54 available for equipping the pen 50.

Referring to FIG. 5A, the pen comprises a sleeve 60 and, in the extension of this sleeve, a head 61. The sleeve and the head form a rectilinear light pipe, described in detail below.

In sleeve 60, the end 61 of the cable 46 reveals the end of the optical fibers 62 provided with a ring 63 fixed by a screw 64 at the inlet of a polarizer 65, fixed to the inside wall of the sleeve by sealing rings, which can be seen in FIG. 5A but have not been given reference numbers.

At the outlet of polarizer 65, in head 61, there is a collar 66 integral with a sliding button 67, the whole being free to rotate about the axis of sleeve 60 and head 61 because of the opening 69 provided in the head (see FIG. 6), in the two directions of rotation L and D. Opening 69 makes it possible to turn the sliding button and the collar through at least a right angle.

A plate 70, notably having the function of filtering and polarizing the light, is fixed in collar 66 by means of two sealing rings, which can be seen but are not referenced. This plate is for example a quarter-wave plate or a half-wave plate. Plate 70 filters the light leaving the polarizer. In position 71, marked D on head 61 of the pen, the light obtained is right-polarized light, and in position 72, marked L, it is left-polarized light. In the middle position 73, the polarized light leaving polarizer 65 is unchanged.

At the outlet of the quarter-wave plate 70 there is an iris 74, which can be adjusted by a second sliding button 75 in a manner identical to that just described. Rotation of the iris notably permits the emitted light beam to be focused, notably in relation to the requirements of the treatment.

Head 61 is, for example, equipped with one of the two tips 53 or 54 shown in FIGS. 5C and 5B respectively. These tips screw onto a thread 80 provided in the front opening of head 59 and on the diameters 81 of the rear openings of the two tips.

The tip includes for example a focusing lens 86.

According to a first embodiment of the equipment, the light source consists on, for example, a halogen lamp and of a set of monochromatic filters which are inserted between the lamp and the cable connection 40, 46. The filters are selected for example for filtering at least the wavelengths of light 400 to 700 nanometers.

According to a second embodiment, the light source consists on, a set of laser diodes of monochromatic light evenly distributed in the visible spectrum and even wider to cover the whole spectrum that can be active.

The equipment according to the invention notably makes it possible to project a light beam onto living tissues and to combine the chrominances and the direction of polarization of the light in relation to the bioelectronic affinity of the living cells and the pathology to be treated.

While remaining within the scope of the invention, the polarizer can be a circular polarizer.

The sliding buttons can be replaced by micro-motors for remote control.

The base can contain all the mechanisms for adjusting the light for the treatment, i.e. the set of laser lamps and the means for selecting them in the light box, or the set of filters and the means for selecting them, the polarizer 65, the quarter-wave plate 70 and its sliding button 67, or its adjusting micro-motor. It is then sufficient to provide an optical-fiber cable 46, the fibers of which are of the type with conservation of polarization.

1. A phototherapy equipment comprising a light source, a light guide suitable for directing the light to the inlet of a terminal pen for projecting a light beam onto living tissues, wherein it comprises at least one plate at the outlet of the polarizer arranged to give the light a defined, clockwise or counterclockwise direction (D, L) of polarization.

2. The equipment as claimed in claim 1, wherein the plate is a quarter-wave plate.

3. The equipment as claimed in claim 1, wherein the plate is a half-wave plate.

4. The equipment as claimed in claim 3, wherein the plate is positioned according to two positions of use.

5. The equipment as claimed in claim 4, wherein the plate is positioned at approximately 45° to the left or at approximately 45° to the right of a neutral position.

6. The equipment as claimed in claim 5, wherein the two positions are obtained under the action of a micro-motor acting in clockwise (D) or counterclockwise (L) rotation.

7. The equipment as claimed in claim 6, wherein the light guide is an optical fiber cable.

8. The equipment as claimed in claim 7, wherein the pen includes an iris.

9. The equipment as claimed in claim 1, wherein it includes a barrel provided with filters, Fi, of different wavelengths connected to a drive unit, the barrel being arranged at the outlet of the light source.

10. The equipment as claimed in claim 9, wherein the polarizer is a circular polarizer.

11. The equipment as claimed in claim 10, wherein the polarizer is an elliptical polarizer.

12. The equipment as claimed in claim 11, wherein the light source is a halogen or xenon lamp equipped with monochromatic filters.

13. The equipment as claimed in claim 12, wherein the light source is a laser diode.

14. The equipment as claimed in claim 1, wherein the light source comprises a set of laser diodes of different colors.

15. A method of cosmetic treatment of tissues of biological cells by phototherapy comprising the irradiation of the tissue with incoherent and/or coherent, polarized monochromatic light, characterized in that the wavelength to be used is selected and the direction of polarization of the light is determined so as to adapt this dextrorotatory or levorotatory orientation to the right or left chirality of the molecules in relation to the treatment to be applied.

16. A method for application in food-processing industry for the treatment of tissues of biological cells by phototherapy comprising the irradiation of the tissue with incoherent and/or coherent, polarized monochromatic light, characterized in that the wavelength to be used is selected and the direction of polarization of the light is determined so as to adapt this dextrorotatory or levorotatory orientation to the right or left chirality of the molecules in relation to the treatment to be applied.

17. The method as claimed in claim 15, wherein a quarter-wave plate is used.

18. The method as claimed in claim 16, wherein a half-wave plate is used.

19. An application of the equipment as claimed in claim 1, wherein the treatment of biological cells by phototherapy comprising the irradiation of the tissue with incoherent and/or coherent, polarized monochromatic light, characterized in that the wavelength to be used is selected and the direction of polarization of the light is determined so as to adapt this dextrorotatory or levorotatory orientation to the right or left chirality of the molecules in relation to the treatment to be applied.
